(19) United States
(12) Patent Application Publication
(10) Pub. No.: US 2009/0157070 A1
(43) Pub. Date: Jun. 18, 2009

(54) MEDICAL TREATMENT USING PULSED ENERGY

(75) Inventors: Christopher L. Oskin, Grafton, MA (US); Isaac Ostrovsky, Wellesley, MA (US)

Correspondence Address: COOLEY GODWARD KRONISH LLP;ATTN: Patent Group, Suite 1100, 777 - 6th Street, NW, WASHINGTON, DC, 20001, US

(73) Assignee: Boston Scientific Scimed, Inc., Maple Grove, MN (US)

(21) Appl. No.: 12/335,073

(22) Filed: Dec. 15, 2008

Related U.S. Application Data
(60) Provisional application No. 61/014,535, filed on Dec. 18, 2007.

(51) Int. Cl. A61B 18/18; A61M 29/02

(52) U.S. Cl. 606/33; 606/192

(57) ABSTRACT

An apparatus and methods for the treatment of tissue includes compressing a portion of the tissue to be treated using an expandable member and emitting a plurality of energy pulses toward that portion of the tissue. The pulses can be pulses of electromagnetic radiation and can be based on a pulse parameter value. The pulse parameter value can be modified based on a signal from a sensor.

### CROSS-REFERENCE TO RELATED CASE

This application claims priority to, and the benefit of Provisional U.S. Patent Application Ser. No. 61/014,535, filed Dec. 18, 2007, the entirety of which is incorporated herein by reference.

### TECHNICAL FIELD

The invention generally relates to treatment of tissue using energy emissions and more particularly to treatment of the tissue using pulsed energy emissions.

### BACKGROUND INFORMATION

When treating a tissue of a patient with thermotherapy, it is often necessary to heat a significant portion of the tissue without damaging healthy portions of the tissue and surrounding tissues. Thermotherapy can be used to treat, for example, the prostate gland, which is a frequently diseased internal organ that encircles the urethra immediately below the bladder. For example, thermotheraphy can be used to treat benign prostatic hyperplasia (BPH), which is a nonmalignant, bilateral nodular tumorous expansion of prostate tissue occurring mainly in the transition zone of the prostate. Left untreated, BPH can cause an obstruction of the urethra that can result in increased urinary frequency, urgency, incontinence, nocturia, and/or a slow or interrupted urinary stream. Even though known thermotherapy techniques/procedures can be effective, they can take considerable periods of time to perform and can result in undesirable thermal damage to nearby tissues not targeted for treatment. Accordingly, methods and apparatus are needed to address the shortfalls of present technology/procedures and to provide other new and innovative features related to thermotherapy.

### SUMMARY OF THE INVENTION

Embodiments of the invention relate to compressing a portion of a tissue of an inner-surface of a lumen defined by a prostate of a patient using an expandable member and emitting a plurality of energy pulses toward that portion of the tissue. The pulses can be pulses of electromagnetic radiation and can be based on a pulse parameter value. The pulse parameter value can be modified based on a signal from a sensor.

In one aspect, the invention generally relates to a method that includes compressing a portion of a tissue of an inner-surface of a lumen defined by a prostate of a patient using an expandable member when the expandable member is in an expanded configuration. A plurality of energy pulses are emitted toward the portion of the tissue.

Embodiments according to this aspect of the invention can have a variety of features and/or additional elements. For example, in some embodiments, a duration of at least one energy pulse from the plurality of energy pulses can be defined based on a feedback signal associated with the compressed portion of the tissue. In some embodiments, a duration of a period between at least two energy pulses from the plurality of energy pulses may be defined based on at least one of a signal from a rectal probe or a signal associated with the compressed portion of the tissue. In some embodiments, the method may also include emitting at least one energy pulse that can have an output power different than an average output power associated with the plurality of energy pulses.

In some embodiments, the method can also include emitting, before the emitting associated with the plurality of energy pulses, at least one energy pulse that has an output power higher than an output power of each of the energy pulses from the plurality of energy pulses. The at least one energy pulse may be configured to ablate at least a portion of the portion of the tissue and the plurality of energy pulses may be configured to cause necrosis.

In some embodiments, the method can also include inserting the expandable member when the expandable member is in the contracted configuration. Each energy pulse from the plurality of energy pulses can be an electromagnetic radiation pulse that has a spectral region corresponding to a microwave. In some embodiments, the plurality of energy pulses can be defined such that a temperature of a rectal wall lateral to the prostate is substantially a normal body temperature of the patient.

In another aspect, the invention generally involves an apparatus that includes an expandable member that has a contracted configuration and an expanded configuration. The expandable member is configured to be inserted into a portion of a lumen defined by a prostate when the expandable member is in the contracted configuration. The expandable member is configured to dilate the portion of the lumen when in the expanded configuration. The apparatus also includes an energy-emitting source that has at least a portion disposed within the expandable member and configured to emit a plurality of energy pulses toward the portion of the lumen when the portion of the lumen is dilated by the expandable member when in the expanded configuration.

Embodiments according to this other aspect of the invention can have a variety of features and/or additional elements. For example, in some embodiments, the apparatus can also include a rectal probe configured to produce a signal corresponding with a temperature of a rectal wall and a control module configured to receive the signal and configured to prevent the energy-emitting source from emitting energy for a period of time when a threshold condition is satisfied based on the signal. In some embodiments, at least one energy pulse from the plurality of energy pulses can have a pulse duration and a power output value defined such that a temperature gradient from a surface of the portion of the lumen to an inner-surface of a rectal wall is transient during the entire pulse duration.

In some embodiments, the expandable member can be a balloon and the apparatus can also include an anchor balloon that has a contracted configuration and an expanded configuration. The energy-emitting source may have a specified position within the lumen when the anchor balloon is in the expanded configuration. In some embodiments, at least a portion of the plurality of energy pulses can be defined based on a feedback signal associated with the portion of the lumen. In some embodiments, the energy-emitting source can be configured to function as an antenna between a pair of energy pulses from the plurality of energy pulses.

In some embodiments, the energy pulse can have an output power greater than 50 watts and the energy pulse has a duration less than 1 second. In some embodiments, the plurality of energy pulses may be defined to cause necrosis of the portion of the lumen. In some embodiments, the expandable member can be changed from the contracted configured to the expanded configuration when a fluid is injected into the expandable member.

In some embodiments, each energy pulse from the plurality of energy pulses may be an electromagnetic radiation pulse that has a spectral region corresponding to a microwave. In some embodiments, the portion of the lumen substantially can surround the energy-emitting source.

In yet another embodiment, the invention generally features a method that includes compressing a portion of a prostatic urethra using an expandable member. An electromagnetic radiation pulse is emitted toward the compressed portion based on a pulse parameter value and the pulse parameter value is modified based on a signal from a sensor. In some embodiments, the signal corresponds with a measurement of the compressed portion and the signal is defined during a rest period after the emitting.

Embodiments according to this additional aspect of the invention can have a variety of features and/or additional elements. For example, in some embodiments, the electromagnetic radiation pulse can be a first electromagnetic radiation pulse that has a spectral region corresponding with a microwave. The method may also include emitting a second electromagnetic radiation pulse based on the pulse parameter value after the modifying. In some embodiments, the indicator can be associated with an impedance of the portion of the prostatic urethra.

In some embodiments, the emitting can include emitting from an electromagnetic radiation source. The signal can be a first signal and at least a portion of the electromagnetic radiation source can be configured to function as the sensor. The method can also include receiving after the emitting a second signal at the portion of the electromagnetic radiation source where the first signal can be defined based on the second signal. In some embodiments, the indicator can be associated with a temperature measurement from a rectal probe. The pulse parameter value can be an output power value and the output power value can be increased based on the indicator.

### BRIEF DESCRIPTION OF THE DRAWINGS

For a more complete understanding of the nature and operation of various embodiments according to the invention, reference is made to the drawings briefly described below and also to the description that follows this brief description of the drawings. The same or similar reference numbers in the drawings generally denote the same or similar elements of the various disclosed embodiments. The drawings are not necessarily to scale, emphasis instead generally being placed on conveying certain concepts and aspects according to the invention.

FIG. 1 is a schematic block diagram that illustrates a thermodilatation system configured to deliver pulses of energy to a target tissue during a medical treatment of a patient, according to an embodiment of the invention.

FIG. 2 is a schematic block diagram of a vertical sectional view of a male pelvic region that includes a transurethral thermodilatation system configured to emit pulses of electromagnetic radiation to a prostate affected by BPH, according to an embodiment of the invention.

FIG. 3 is a schematic graph that illustrates a profile of pulses that can be produced by an energy-emitting source, according to an embodiment of the invention.

FIG. 4 illustrates a table of pulse parameter values that can be used to define one or more pulses emitted from an energy-emitting source, according to an embodiment of the invention.

FIG. 5A is a schematic diagram that illustrates temperature profiles associated with an energy pulse shown in FIG. 5B, according to an embodiment of the invention.

FIG. 5B is a schematic graph that illustrates an energy pulse, according to an embodiment of the invention.

FIG. 6 is a schematic diagram that illustrates a control module configured to define energy emitted from an energy-emitting source using a control loop module, according to an embodiment of the invention.

FIG. 7 is a schematic diagram that illustrates energy pulses defined based on a feedback signal, according to an embodiment of the invention.

FIG. 8 is a flowchart that illustrates a method for modifying an energy pulse emitted from an energy-emitting source, according to an embodiment of the invention.

FIG. 9 is a schematic diagram that illustrates a set of energy pulses that can be emitted towards a target tissue, according to an embodiment of the invention.

### DESCRIPTION

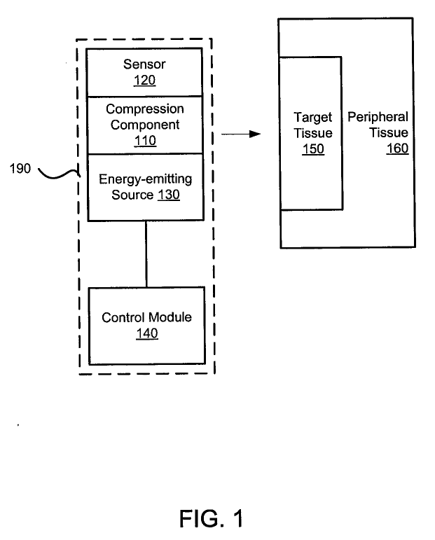

FIG. 1 is a schematic block diagram that illustrates a thermodilatation system 190 configured to deliver pulses of energy to a target tissue 150 during a medical treatment of a patient, according to an embodiment of the invention. The pulses of energy and time between pulses of energy are defined to allow for heat dissipation so that undesirable thermally-related damage to the target tissue 150 can be substantially avoided. The pulses of energy are also defined to allow for heat dissipation between pulses of energy so that undesirable thermally-related damage to peripheral tissue 160, which can be proximate to or adjacent to the target tissue 150, can be substantially avoided. A pulse of energy can have, for example, an output power of approximately 70 watts and a duration of approximately 100 milliseconds.

Although the energy-emitting source 130 can be configured to emit a sustained level of energy to treat the target tissue 150 during a medical procedure, pulses of energy can be emitted by the energy-emitting source 130 in lieu of, and/or in addition to, the sustained level of energy such that the overall treatment time of the target tissue 150 during the medical procedure can be reduced (e.g., <1 percent to 90 percent). In some embodiments, the overall treatment time can be reduced significantly while achieving substantially the same or improved results. In some embodiments, the overall treatment time can be increased while achieving substantially the same or improved results. In some embodiments, for example, pulses of energy can be defined to deliver a higher output power than can be used in a comparable sustained energy delivery treatment because the cycles of energy pulses allow for significant heat dissipation. The use of the higher output power can result in a shorter overall treatment time of the target tissue 150.

The target tissue 150 and/or the peripheral tissue 160 can be associated with, for example, an organ of a patient. The thermodilatation system 190 can be configured to treat target tissue 150 that is adjacent to a bodily conduit (not shown). For example, the thermodilatation system 190 can be configured to treat a prostatic urethra that is undesirably constricted due to enlargement of a prostate of a patient—a condition referred to as benign prostatic hyperplasia (BPH). This system, or variations thereof, can be used to treat other conditions such as female stress incontinence (FSI) and gastro-esophagus reflux diseases (GERD), and/or a tumor (e.g., breast cancer tumor).

The thermodilatation system 190 shown in FIG. 1 has a sensor 120, a compression component 110, an energy-emitting source 130, and a control module 140. The control module 140 is configured to control each of the components of the thermodilatation system 190 during, for example, a medical procedure to treat a prostatic urethra affected by BPH. The compression component 110 is configured to compress a portion of the target tissue 150, and the energy-emitting source 130 is configured to emit pulses of energy towards the target tissue 150.

In some embodiments, the energy-emitting source 130 can be configured to emit a spectral region of electromagnetic radiation associated with a radio wave, a microwave, terahertz radiation, infrared radiation, visible light, ultraviolet radiation, an x-ray, and/or a gamma ray. The energy-emitting source 130 source can be configured to deliver sustained/constant energy (e.g., electromagnetic radiation) for a specified period of time (e.g., 20 minutes) as well as one or more pulses of energy that can have specified power output levels and/or durations. In some embodiments, the energy-emitting source 130 can be, for example, a microwave power oscillator or a laser (e.g., a gain medium with a resonant optical cavity).

The sensor 120 can be configured to measure, for example, a temperature and/or an impedance that can be used in one or more control loops to reduce or prevent undesirable side-effects related to use of the thermodilatation system 190. For example, the sensor 120 can be configured to measure an impedance of the target tissue 150 and/or a temperature associated with the peripheral tissue 160. The impedance of a tissue can be an indicator of an ability of the tissue to conduct heat (e.g., high impedance can be inversely proportional to heat conductivity).

In some embodiments, the temperature and/or impedance measured by the sensor 120 can be used to define a function of the thermodilatation system 190 (e.g., used to define/modify an energy pulse emitted from the electromagnetic radiations source 130). The sensor 120 can be any type of sensor that can detect a property (e.g., a temperature) and send a signal corresponding to the property. For example the sensor 120 can be an electrical resistance thermometer, a thermocouple, an infrared thermometer, an antenna, a microphone, and/or an image detecting device. In some embodiments, the energy-emitting source 130 can also function as a sensor in addition to functioning as an energy emitter. For example, the energy-emitting source 130 can function as an antenna that receives a signal from the target tissue 150 between emitted energy pulses.

The control module 140, which can be one or more software and/or hardware modules, can be configured to send signals to and/or receive signals from each of the components within the thermodilatation system 190. The control module 140 can be configured to control (e.g., trigger/invoke a function of) each of the components of the thermodilatation system 190 via wireless communication signals and/or wired communication signals. The control module 140 can be configured to control each of the components based on a sequence of instructions associated with a medical procedure. The control module 140 can be configured to control various functions related to, for example, treatment setup, treatment control, patient treatment file storage and retrieval, hardware diagnostics, hardware system monitoring for the energy-emitting source 130, thermometry, fluid warming and pumping system.

In some embodiments, the control module 140 can be included in an instrument cabinet that can be, for example, a mobile unit. In some embodiments, portions of the control module 140 and/or functions associated within the control module 140 can be distributed can be included in different portions of the thermodilatation system 190.

During a medical procedure, for example, the compression component 110 can be configured to compress a portion of the target tissue 150 while the energy-emitting source 130 emits energy, such as pulses of energy, towards the compressed target tissue 150. The sensor 120 can be used to measure a temperature of the peripheral tissue 160 as the peripheral tissue 160 conducts heat from the compressed target tissue 150 when the compressed target tissue 150 is being treated. The temperature can be used in a feedback loop to modify the energy emitted from the energy-emitting source 130 to, for example, more effectively treat the target tissue 150 and/or to ensure that the peripheral tissue 160 is not thermally damaged during the medical procedure. The function(s) associated with energy-emitting source 130 and/or the sensor 120 can be controlled by the control module 140 during the medical procedure.

In some embodiments, the thermodilatation system 190 can be a transurethral thermodilatation system 190. For example, the energy-emitting source 130 can be configured to treat the target tissue 150 (e.g., a tissue associated with a prostate of a patient) via a urethra of a patient. In some embodiments, a transurethral thermodilatation system that has a microwave emitter can be referred to as a transurethral microwave therapy (TUMT) device/system.

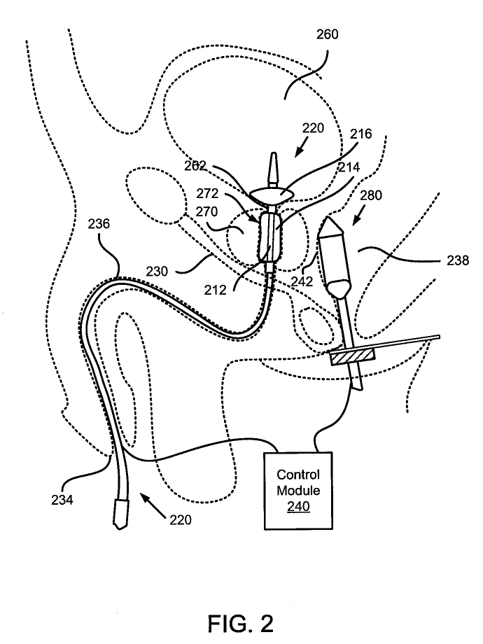

FIG. 2 is a schematic block diagram of a vertical sectional view of a male pelvic region that includes a transurethral thermodilatation system configured to emit pulses of electromagnetic radiation to a prostate 270 affected by BPH, according to an embodiment of the invention. The transurethral thermodilatation system shown in FIG. 2 includes a catheter 220, a control module 240, and a rectal monitor 280. In some embodiments, the rectal monitor 280 can be a rectal thermal monitor.

The catheter 220 is configured to be inserted into the male pelvic region via a urethra 236, which is a duct leading from bladder 260, through prostate 270 and out orifice 234. In some embodiments, the catheter 220 can also be referred to as a transurethral catheter. As shown in FIG. 2, the catheter 220 has an expandable member 214 and an anchoring balloon 216 (e.g., Foley balloon) that are both in an expanded configuration (e.g., inflated configuration). In some embodiments, the expandable member 214 and/or the anchoring balloon 216 can be in their respective expanded configurations during one or more portions of a medical procedure. In some embodiments, the expandable member 214 is configured to be inserted via the urethra 236 into the prostate 270 when in a contracted configuration (not shown). Likewise, in some embodiments, the anchoring balloon 216 is configured to be inserted via the urethra 236 into the bladder 260 when in a contracted configuration (not shown).

The catheter 220 includes an electromagnetic radiation source 212 disposed within the expandable member 214. The electromagnetic radiation source 212 can be configured to emit electromagnetic radiation towards the prostatic urethra 272 to substantially reduce or eliminate constriction of the prostatic urethra 272 (not shown because expandable member 214 is in an expanded configuration) caused by, for example, benign tumorous tissue (not shown) within the prostate 270. The electromagnetic radiation source 212 can be configured to dilate the prostatic urethra 272 by heating and causing necrosis of the encroaching benign tumorous tissue. The electromagnetic radiation source 212 can be configured to emit electromagnetic radiation at a variety of output levels and/or time periods towards the prostatic urethra including, for example, pulses of electromagnetic radiation such that the constriction can be reduced and urine can flow from bladder 260 to orifice 234 with less interruption. In some embodiments, periurethral tumorous tissue of prostate 270 anterior and lateral to prostatic urethra 272 is heated and/or killed while avoiding unnecessary and undesirable damage to, for example, urethra 236 and to proximate healthy tissues, such as external sphincter 230, rectum 238, and bladder neck 262.

The electromagnetic radiation source 212 can be configured to emit pulses of electromagnetic radiation when the prostatic urethra 272 is being dilated and the wall (e.g., inner surface) of the prostatic urethra 272 is compressed by the expandable member 214. By emitting pulses of electromagnetic radiation towards the prostatic urethra 272 when the prostatic urethra 272 is dilated/compressed, the prostatic urethra 272 can be changed to a substantially dilated position (e.g., unconstricted position) even after the catheter 220 is removed from the male pelvic region. For example, the prostastic urethra 272 can be fixed in a dilated position when at least a portion of the prostatic urethra 272 is heated/killed by energy pulses while being dilated/compressed. In some embodiments, the electromagnetic radiation source 212 can be configured to emit pulses of electromagnetic radiation any time before or after the expandable member 214 is expanded. The electromagnetic radiation source 212 can be triggered by the control module 240 to emit electromagnetic radiation as pulses.

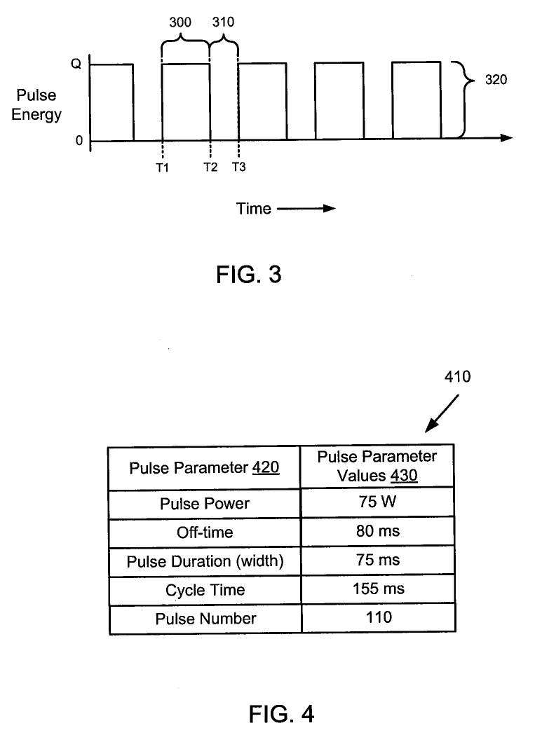

For example, FIG. 3 is a schematic graph that illustrates a profile of pulses that can be produced by an energy-emitting source such as electromagnetic radiation source 212 shown in FIG. 2, according to an embodiment of the invention. The y-axis of the graph illustrates pulse energy and the x-axis of the graph illustrates time increasing to the right.

As shown on the y-axis, each of the pulses in the graph have a power of Q (represented by height 320). The power of the pulses can be referred to as an output power or as an amplitude. In some embodiments, the output power of the pulse can be, for example, between 0 watts and 300 watts. Each of the pulses shown in this graph have a duration 300 (shown as a period of time between time T1 and time T2) and an off-time 310 between pulses (shown as a period of time between time T2 and time T3). In some embodiments, the duration of the pulse 300 can be referred to as a pulse width and the off-time 310 can be referred to as a rest period or heat dissipation period. In some embodiments, the pulse width and/or off-time can be, for example, between a fraction of a millisecond (e.g., 0.2 milliseconds) and several seconds (e.g., 10 seconds, 2 minutes). The pulse duration 300 and subsequent off-time time 310 can collectively be referred as a pulse cycle and can have a cycle time between time T1 and T3.

During a medical treatment, the pulses of energy can be emitted towards the prostatic urethra 272 (shown in FIG. 2). The duration of the pulses 310 can be defined so that at least a portion of the prostatic urethra 272 is heated to cause necrosis of the portion. The off-time 310 between the pulses can be defined so that at least a portion of the heat generated at and/or conducted at the prostatic urethra 272 and/or surrounding tissue during the energy pulse 300 can be dissipated by, for example, blood flow or other conduction mechanisms. The heat dissipated during the off-times (such as off-time 310) between pulses can substantially reduce damage that can be caused to, for example, tissue proximate to the prostatic urethra 272 such as the rectum 238. More details related to electromagnetic radiation pulse emissions from an electromagnetic radiation source and the functionality of the control module are discussed in connection with FIGS. 4 through 10.

Referring back to FIG. 2, in some embodiments, the electromagnetic radiation source 212 can be located within the expandable member 214 during treatment of the prostate 270 such that the electromagnetic radiation source 212 is substantially centrally located within the prostatic urethra 272. In some embodiments, the expandable member 214 and/or the electromagnetic radiation source 212 can be configured so that the electromagnetic radiation source 212 is biased, for example, towards a portion (e.g., a side wall) of the prostatic urethra 272 such that a higher concentration of electromagnetic radiation is emitted towards a particular location within the prostatic urethra 272. In some embodiments, the electromagnetic radiation source 212 can be configured to emit electromagnetic radiation to selectively treat (e.g., heat) some portions of the prostatic urethra 272.

In some embodiments, the electromagnetic radiation source 212 can be configured to ablate one or more portions of the prostate 270, such as the prostatic urethra 272, during a medical procedure. More details related to ablation of at least a portion of the prostate 270 during a medical procedure are discussed in connection with FIG. 9.

In some embodiments, the expandable member 214 can be configured such that a fluid (e.g., warmed fluid, cooling fluid) can be injected into the expandable member 214 to control, for example, the temperature of the prostatic urethra 272 walls adjacent to the expandable member 214. The fluid can be injected via a lumen within the catheter 220. In some embodiments, a fluid (e.g., warmed fluid) can be used to change the expandable member 214 and/or the anchor balloon 216 from a contracted configuration into an expanded configuration. In some embodiments, the fluid is below a body temperature of a patient (e.g., approximately 30° C.) before being injected into the expandable member 214 and/or anchor balloon 216. In some embodiments, the fluid used to inflate expandable member 214 can be configured to conduct electromagnetic radiation (e.g., a minimally energy absorptive solution) to more efficiently heat the prostatic urethra 272. In some embodiments, the fluid can be constantly or periodically circulated through the expandable member 214. In some embodiments, the fluid can be circulated as needed through the expandable member 214.

The rectal monitor 280, which is configured to be inserted into the rectum 238, can have, for example, one or more thermal sensors (not shown). The thermal sensors can be configured to measure the amount of heat from absorption of electromagnetic radiation emitted by the electromagnetic radiation source 212. The heat can be manifested by and measured as a temperature at the rectal wall 242. In some embodiments, the rectal monitor 280 can be configured to detect a temperature substantially lateral to the prostate 270. The temperature at the rectal wall 242 can be an indicator of a dangerous heat level caused by electromagnetic emissions from the electromagnetic radiation source 212. In some embodiments, for example, the rectal monitor 280 can be have two or more thermal sensors that can be integrally mounted at differing radial locations on the probe and spaced, for example, approximately 1 centimeter from one another.

In some embodiments, the rectal monitor 280 can be configured to send a signal that corresponds with one or more temperatures to the control module 240. The control module 240 can process the signal at a processor (not shown) and can terminate (e.g., prevent) one or more emissions from the electromagnetic radiation source 212 if the temperature at the rectal wall satisfies a threshold condition. For example, the control module 240 can be configured to cut-off power to the electromagnetic radiation source 212 if the temperature at the rectal wall exceeds a threshold limit of 43° C.

In some embodiments, one or more sensors (not shown) of the rectal monitor 280 can be configured to perform measurement functions (as triggered by, for example, the control module 240), during different portions of a pulse cycle associated with the electromagnetic radiation source 212. For example, a sensor (e.g., temperature sensor) of the rectal monitor 280 can be configured to measure a temperature (or a different property) during a rest period, and the same sensor (and/or a different sensor) can be used to measure a temperature during an energy pulse emitted from the energy emitting source 212. In some embodiments, the rectal monitor 280 (e.g., a sensor of the rectal monitor 280) can be configured to only take measurements during the rest period or during the energy pulse. In some embodiments, the rectal monitor 280 can be configured to take measurements only during certain rest periods and/or energy pulses.

In some embodiments, the rectal monitor 280 can be configured to achieve a variety of functions. For example, the rectal monitor 280 can be configured to detect electromagnetic radiation levels using an electromagnetic radiation sensor (not shown) and can be configured to send a signal associated with the detected level to the control module 240. In some embodiments, the rectal monitor 280 can be referred to as a rectal probe.

The anchor balloon 216 can be used to place the electromagnetic radiation source 212 and/or expandable member 214 into a desirable location within the prostate 270. The anchor balloon 216, for example, can be inserted into the bladder 260 so that the proximal end of the electromagnetic radiation source 212 and/or expandable member 214 are located at the prostate 270 immediately distal of the bladder neck 262. In some embodiments, x-ray and/or ultrasound techniques can be used to determine the location of or can be used to place the electromagnetic radiation 212 within the male pelvic region.

The length of expandable member 214 can vary depending upon the size of the prostate 270. A typical length of the expandable member 214 can be about 40 millimeters. In some embodiments, the length of the expandable member 214 can range from 25 to 60 millimeters.

In some embodiments, the distal end portion of catheter 220 can be approximately 18 French (French is a measurement equal to 0.333 mm or 0.013 inch). Since the average diameter of the urethra 236 of a male adult human is approximately 22 French, the contracted expandable member 214 that surrounds the catheter 220 can be approximately 2 French so that diameter of catheter 220 and balloon 214 would be less than that of the patient's urethra 236 for ease of insertion and less pain for the patient. In some embodiments, the catheter 220 can include multiple lumen shafts (not shown) for injecting fluids and/or therapeutic agents.

In some embodiments, the electromagnetic radiation source 212 can be configured to emit different pulses of electromagnetic radiation (e.g., different output levels, different durations) depending on the expansion of the expandable member 214. For example, the electromagnetic radiation source 212 can be configured to emit a first type of pulse when the expandable member 214 is a in a first expanded configuration and can be configured to a emit a second type of pulse when the expandable member 214 is in a second expanded configuration.

FIG. 4 illustrates a table 410 of pulse parameter values 430 that can be used to define one or more pulses emitted from an energy-emitting source, according to an embodiment of the invention. The table 410 illustrates pulse parameters 420 and corresponding pulse parameter values 430. The fields illustrated in the table 410 can, in some embodiments, be included in a memory such as a database that can be accessed by a control module. The control module can use, for example, the pulse parameter values 430 to trigger emission of a pulse from an energy-emitting source.

The pulse parameters 420 illustrated in the table are pulse power, off-time, pulse duration (width), cycle time, and pulse number. The pulse number is an indicator of the number of pulse cycles (or pulses) to be performed/emitted during a particular medical procedure. In some embodiments, a pulsing duration can be specified in addition to, or in place of, a pulse number. The pulse parameters 420 and associated pulse parameter values 430 shown in FIG. 4, can collectively be referred to as an energy pulse definition.

In some embodiments, a library of energy pulse definitions such as that shown in FIG. 4 can be included in a database (e.g., a local memory, a remote memory) that can be accessed by a control module. Each of the energy pulse definitions can be selected and/or implemented based on, for example, a medical procedure type, a tissue type, and/or a clinical requirement. For example, a first type of energy pulse definition can be used during a medical procedure if a tissue has a first level of severity, and a second type of energy pulse definition can be used during the medical procedure if the tissue has a second level of severity.

The pulse duration, the off-time, and/or the pulse power of one or more pulses can be defined based on a sensitivity, a heat dissipation characteristic, and/or a conduction consideration of a target tissue and/or of a peripheral tissue. For example, if a peripheral tissue adjacent to a target tissue is relatively unsusceptible to thermally-related damage and/or the rate of heat dissipation from the peripheral tissue is relatively fast, the off-time between emitted energy pulses can be shorter than if the peripheral tissue were susceptible to thermally-related damage and/or the heat dissipation were relatively slow. Likewise, if the peripheral tissue is relatively unsusceptible to thermally-related damage and/or the rate of heat dissipation from the peripheral tissue is relatively fast, the amplitude of the energy pulses and/or duration of the energy pulses can be greater than if the peripheral tissue were susceptible to thermally-related damage and/or the heat dissipation were relatively slow. In some embodiments, a definition of one or more pulses can be defined based on, for example, an impedance of a target tissue, a robustness of the target tissue to energy pulses, and/or a proximity of the target tissue to a peripheral tissue.

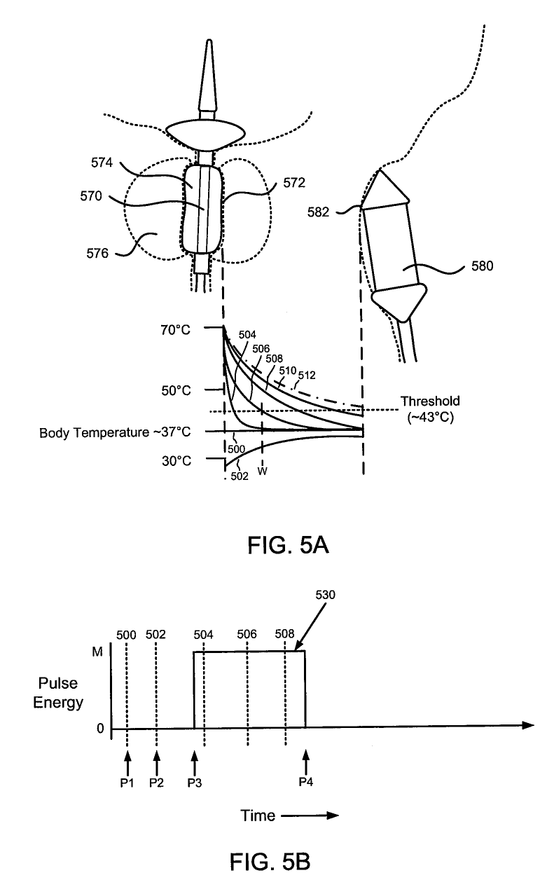

FIG. 5A is a schematic diagram that illustrates temperature profiles associated with an energy pulse 530 shown in FIG. 5B, according to an embodiment of the invention. Specifically, FIG. 5A illustrates a set of temperature profiles 500-512 between a prostatic urethra 572 and a rectal wall 582, and FIG. 5B illustrates the relationship of the temperature profiles 500-512 with the energy pulse 530. FIGS. 5A and 5B illustrate the transient nature of the temperature profile between the prostatic urethra 572 and the rectal wall 582 during the medical procedure when the energy pulse 530 is emitted by an energy-emitting source 570 towards the prostatic urethra 572. In this embodiment, the temperature of the rectal wall 582 is measured by temperature probe 580 (e.g., rectal thermal monitor, rectal probe) is substantially lateral to the prostatic urethra 572. In some embodiments, a temperature profile can be referred to as a temperature gradient. The energy pulse 530 can be one pulse from a plurality of energy pulses (not shown).

The temperature profile 500 (shown in FIG. 5A) is substantially flat at approximately a body temperature of a patient (˜37° C.) at time P1 before a medical procedure is initiated (shown in FIG. 5B). In this embodiment, the temperature at the prostatic urethra 572 decreases, resulting in temperature profile 502 (shown in FIG. 5A), when a cooling fluid is injected into an expandable member 574 at time P2 (shown in FIG. 5B).

During emission of the energy pulse 530 at output power level M between times P3 and P4, the temperature at the prostatic urethra 572 rises to approximately 70° C. (shown in FIG. 5A) and the temperature profile changes from 504 to 506, and from 506 to 508 (shown in FIG. 5B). The temperature profile 504, which is near the beginning of the energy pulse 530, is steeper near the prostatic urethra 572 than, for example, the temperature profile 508, which is near the end of the energy pulse 530. For convenience, the temperature profile at only three different points during the emission of the energy pulse 530 are shown in FIGS. 5A and 5B. In some embodiments, the temperature profile between the prostatic urethra 572 and the rectal wall 582 can change smoothly, steadily, and/or rapidly.

In this embodiment, the energy pulse 530 is defined such that the temperature at the rectal wall 582 is substantially constant during the energy pulse 530. Also, in this embodiment, the temperature profile between the prostatic urethra 572 and the rectal wall 582 is transient during the entire duration of the energy pulse 530. In some embodiments, the duration and/or output power of the energy pulse 530 can be defined such that the temperature profile between the prostatic urethra 572 and the rectal wall 582 is substantially transient during the entire duration of the energy pulse 530, and subsequent energy pulses, if any. Because the temperature profile near the prostatic urethra 572 is steep and transient during the energy pulse 530, the temperature between, for example, point W and the rectal wall 582 is below the threshold temperature limit during a significant portion of the emission time period of the energy pulse 530.

In some embodiments, the duration and/or output power of the energy pulse 530 can be defined so that the temperature of the rectal wall 582 increases as shown in, for example, temperature profile 510. FIG. 5A also illustrates a hypothetical temperature profile 512 that could occur if the duration of the pulse 530 were increased beyond that shown in FIG. 5B. A control module can be configured to turn-off the energy-emitting source 570 because the temperature at the rectal wall 582 increases beyond the 43° C. threshold limit at temperature profile 512.

Although not shown, after the emission of the energy pulse 530 (after time P4 shown in FIG. 5B), the temperature at the prostatic urethra 572 can decrease, and the temperature profile can change accordingly. The temperature profile can change, for example, as blood flow between the prostatic urethra 572 and the rectal wall 582 removes heat conducted during the energy pulse 530. In some embodiments, the temperature profile can change, for example, as heat is otherwise conducted away from the region between the prostatic urethra 572 and the rectal wall 582.

In some embodiments, the temperature probe 580 can include a cooling component (not shown). The cooling component can be configured to maintain the temperature of the rectal wall 582 below a specified temperature. For example, a cooling fluid can be circulated (e.g., continuously circulated) through the cooling component to maintain the temperature of the rectal wall 582 below for example, 37° C. during energy pulses such as energy pulse 530. In some embodiments, the cooling component can be configured to adjust the rate of cooling in response to energy pulses. For example, a cooling fluid can be circulated through the cooling component during one or more energy pulses and not circulated during a rest period between energy pulses, and vice versa.

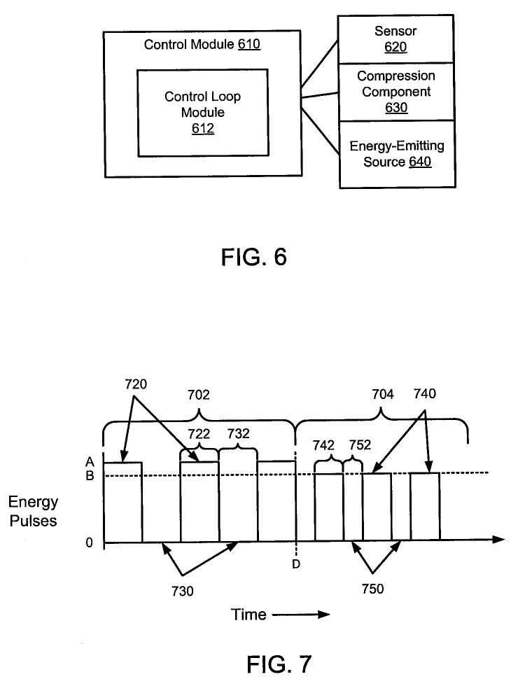

FIG. 6 is a schematic diagram that illustrates a control module 610 configured to define energy emitted from an energy-emitting source 640 using a control loop module 612, according to an embodiment of the invention. The control module 610 can be configured to define and/or modify, for example, one or more energy pulses while they are being emitted and/or one or more energy pulses queued for emission from the energy-emitting source 640 during a medical procedure. In some embodiments, the energy-emitting source 640 can be an electromagnetic radiation source such as a microwave oscillator. The energy-emitting source 640 can be configured to emit energy towards a target tissue (not shown) when the target tissue is being compressed by a compression component 630.

The control loop module 612 of the control module 610 can be used to define and/or modify a pulse parameter value of a pulse being emitted or will be emitted based on, for example, a signal (e.g., feedback signal) from a sensor 620. The signal can be a signal associated with a temperature of a tissue (not shown) proximate to the target tissue or an impedance of the target tissue. For example, the control loop module 612 can receive a temperature signal from the sensor 620 and can be configured to trigger the energy-emitting source 640 to modify an energy pulse currently being emitted from the energy-emitting source 640. In some embodiments, the control loop module 612 can be, for example, any combination of a feedback control module and a feed-forward control module. The control loop module 612 can be associated with an algorithm executed at a processor (not shown) and/or stored in a memory (not shown).

FIG. 7 is a schematic diagram that illustrates energy pulses defined based on a feedback signal, according to an embodiment of the invention. As shown in FIG. 7, energy pulses 720 having an output power of A, duration 722, and off-time 732 are emitted during a time period 702, and energy pulses 740 having an output power of B, duration 742, and off-time 752 are emitted during a time period 704. The energy pulses 740 during time period 704 are defined based on a feedback signal associated with time period 702. For example, the feedback signal can be associated with a temperature of a tissue measured during a energy pulse of time period 702 and/or a temperature of a tissue measured during an off-time between energy pulses of time period 702. As shown in FIG. 7, the change in the energy pulses occurs at time D.

In this embodiment, the duration, output power, and off-time are changed, but in some embodiments, only a few of the pulse parameter values can be modified in response to a feedback signal. For example, the pulse parameter values of the duration and output power can be modified without the off-time being modified.

In some embodiments, the change in the energy pulses at time D can be in response to a feedback signal from a sensor. For example, the change from the energy pulses 720 to the energy pulses 740 can be in response to a temperature sensor. In some embodiments, the change in the energy pulses at time D can be at a specified time after a feedback signal from a sensor has been received.

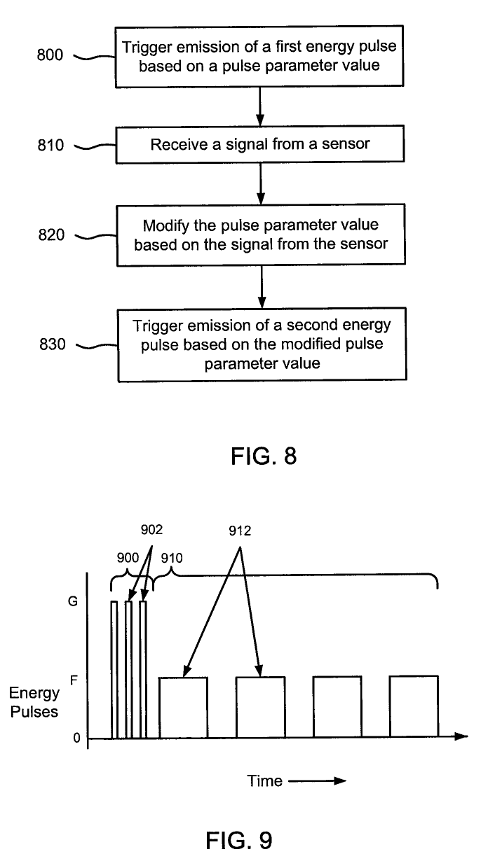

FIG. 8 is a flowchart that illustrates a method for modifying an energy pulse emitted from an energy-emitting source, according to an embodiment of the invention. The flowchart illustrates that emission of a first energy pulse is triggered based on a pulse parameter value at 800. The first energy pulse can be emitted from an energy-emitting source and/or can be triggered by a control module associated with the energy-emitting source. In some embodiments, the pulse parameter value can be stored in, for example, a database that can be accessed by the control module associated with the energy-emitting source. The pulse parameter value can be, for example, a duration, an output power, or an off-time.

A signal is received from a sensor at 810. The signal can be received after, before, and/or during the energy pulse. In some embodiments, the signal can be received at, for example, a control module. The signal can be associated with, for example, a temperature of a tissue (e.g., peripheral tissue) or an impedance of a tissue (e.g., target tissue).

The pulse parameter value is modified based on the signal from the sensor at 820. The pulse parameter value can be modified at, for example, a database that can be accessed by a control module associated with an energy-emitting source. In some embodiments, the pulse parameter value can be calculated based on a mathematical relationship that, for example, includes a value associated with the signal as a variable. In some embodiments, a different pulse parameter value can be selected from a library of pulse parameter values based on the signal. In some embodiments, a pulse parameter value within a program (e.g., sequence of instructions) associated with a medical procedure can be modified based on the signal.

Emission of a second energy pulse is triggered based on the modified pulse parameter value at 830. In some embodiments, the second energy pulse can be triggered based on a schedule defined and used to trigger emission of the first energy pulse. In some embodiments, a number of pulses can be determined based on a feedback signal. For example, a number of pulses typically triggered during a medical procedure can be decreased based on a feedback signal indicating that a temperature of a peripheral tissue is higher than expected. A number of pulses typically triggered during a medical procedure can be increased based on a feedback signal (e.g., visual picture, impedance measurement) indicating that a target tissue requires further treatment.

FIG. 9 is a schematic diagram that illustrates a set of energy pulses that can be emitted towards a target tissue, according to an embodiment of the invention. As shown in FIG. 9, energy pulses 902 are emitted during time period 900 and energy pulses 912 are emitted during time period 910. Each of the energy pulses 902 is a first type of energy pulse defined to ablate the target tissue, and each of the energy pulses 912 is a second type of energy pulse defined to heat the target tissue and cause necrosis without ablating the target tissue. The first type of energy pulses 902 can be referred to as ablating energy pulses 902 and the second type of energy pulses 912 can be referred to as necrosis-inducing energy pulses 912. To accomplish these different objectives, the first type of energy pulses 902 have an output power level that is higher than that of the second type of energy pulses 912. If the energy-emitting source is a microwave-emitting source, the ablating energy pulses 902 can have an output power of more than 100 watts, and the necrosis-inducing energy pulses 912 can have a substantially lower output power (e.g., average output power) of approximately 75 watts.

As shown in FIG. 9, the first type of energy pulses 902 have a duration that is shorter than that of the second type of energy pulses 912. Both energy pulses 902 and energy pulses 912 are defined to have durations that will substantially prevent thermally-related damage to a peripheral tissue proximate to the target tissue. For example, the ablating energy pulses 902 can have a duration of a few milliseconds and an output power of more than 100 watts, and the necrosis-inducing energy pulses 912 can have a substantially longer duration.

As shown in FIG. 9, the ablating energy pulses 902 are emitted towards the target tissue before the necrosis-inducing energy pulses 912. The ablating energy pulses 902 are emitted before the necrosis-inducing energy pulses 912 to remove some of the target tissue at, for example, the beginning of a medical procedure. If the target tissue were, for example, a prostatic urethra that is constricted due to BPH, portions of the prostatic urethra could be ablated using ablating energy pulses 902 before the necrosis-inducing energy pulses 912 are emitted so that the prostatic urethra is dilated at the beginning of a medical procedure to treat the BPH condition.

In some embodiments, the target tissue can be compressed by, for example, an expandable member before, after, and/or during the ablating energy pulses 902 and/or the necrosis-inducing energy pulses 912 are emitted toward the target tissue. For example, the target tissue can be compressed by an expandable member before the ablating energy pulses 902 are emitted towards the target tissue. After the ablating energy pulses 902 have been emitted, and some of the target tissue removed, the expandable member can be further expanded before and/or during the necrosis-inducing energy pulses 912 are emitted.

In some embodiments, the number of necrosis-inducing energy pulses 912 during a medical procedure can be defined based on the number of ablating energy pulses 902. For example, the number of necrosis-inducing energy pulses 912 typically triggered during a medical procedure can be reduced when the number of ablating energy pulses 902 typically triggered during the medical procedure is increased. Likewise, the number of necrosis-inducing energy pulses 912 typically triggered during a medical procedure can be increased when the number of ablating energy pulses 902 typically triggered during the medical procedure is decreased.

In some embodiments, additional ablating energy pulses (not shown) can be performed after the necrosis-inducing energy pulses 912. The additional ablating energy pulses can be performed to assist in setting the tissue in a dilated fashion after the necrosis-inducing energy pulses 912 have been triggered. In some embodiments, ablating energy pulses can be emitted towards a target tissue at any time during a medical procedure.

Some embodiments disclosed herein involve a computer processor and/or computer storage or memory. The computer storage or memory can be a computer-readable medium (also sometimes referred to as a processor-readable medium). The medium can have stored on or in it data and/or instructions or computer code for performing various computer-implemented operations. Examples of computer-readable media include but are not limited to: magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media; carrier wave signals; and hardware devices that are specially configured to store and execute program code, such as application specific integrated circuits (ASICs), Programmable Logic Devices (PLDs), and ROM and random-access memory (RAM) devices. Examples of computer code include but are not limited to micro-code or micro-instructions, machine instructions, such as produced by a compiler, and files containing higher-level instructions that are executed by a computer using an interpreter. For example, an embodiment of the invention may be implemented using Java, C++, or other object-oriented programming language and development tools. Additional examples of computer code include but are not limited to control signals, encrypted code, and compressed code. A computer processor can be a microprocessor or other type of electronic or other type of computing device.

In conclusion, among other things, methods and apparatus for treatment of the tissue of a patient (such as a human or other mammal) using pulsed energy emissions are described. While various embodiments have been described above, it should be understood that they have been presented by way of example only, and various changes in form and details may be made without departing from the spirit or scope of the invention.

What is claimed is:

1. A method, comprising:
compressing a portion of a tissue of an inner-surface of a lumen defined by a prostate of a patient using an expandable member when the expandable member is in an expanded configuration; and
emitting a plurality of energy pulses toward the portion of the tissue.

2. The method of claim 1, wherein a duration of at least one energy pulse from the plurality of energy pulses is defined based on a feedback signal associated with the compressed portion of the tissue.

3. The method of claim 1, wherein a duration of a period between at least two energy pulses from the plurality of energy pulses is defined based on at least one of a signal from a rectal probe or a signal associated with the compressed portion of the tissue.

4. The method of claim 1, further comprising:
emitting at least one energy pulse having an output power different than an average output power associated with the plurality of energy pulses.

5. The method of claim 1, further comprising:
emitting, before the emitting associated with the plurality of energy pulses, at least one energy pulse having an output power higher than an output power of each of the energy pulses from the plurality of energy pulses, the at least one energy pulse configured to ablate at least a portion of the portion of the tissue, the plurality of energy pulses configured to cause necrosis.

6. The method of claim 1, further comprising:
inserting the expandable member when the expandable member is in the contracted configuration, each energy pulse from the plurality of energy pulses is an electromagnetic radiation pulse that has a spectral region corresponding to a microwave.

7. The method of claim 1, wherein the plurality of energy pulses are defined such that a temperature of a rectal wall lateral to the prostate is substantially a normal body temperature of the patient.

8. An apparatus, comprising:
an expandable member having a contracted configuration and an expanded configuration, the expandable member configured to be inserted into a portion of a lumen defined by a prostate when the expandable member is in the contracted configuration, the expandable member configured to dilate the portion of the lumen when in the expanded configuration; and
an energy-emitting source having at least a portion disposed within the expandable member and configured to emit a plurality of energy pulses toward the portion of the lumen when the portion of the lumen is dilated by the expandable member when in the expanded configuration.

9. The apparatus of claim 8, further comprising:
a rectal probe configured to produce a signal corresponding with a temperature of a rectal wall; and
a control module configured to receive the signal and configured to prevent the energy-emitting source from emitting energy for a period of time when a threshold condition is satisfied based on the signal.

10. The apparatus of claim 8, wherein at least one energy pulse from the plurality of energy pulses has a pulse duration and a power output value defined such that a temperature gradient from a surface of the portion of the lumen to an inner-surface of a rectal wall is transient during the entire pulse duration.

11. The apparatus of claim 8, wherein the expandable member is a balloon,
the apparatus further comprising:
an anchor balloon having a contracted configuration and an expanded configuration, the energy-emitting source having a specified position within the lumen when the anchor balloon is in the expanded configuration.

12. The apparatus of claim 8, wherein at least a portion of the plurality of energy pulses is defined based on a feedback signal associated with the portion of the lumen.

13. The apparatus of claim 8, wherein the energy-emitting source is configured to function as an antenna between a pair of energy pulses from the plurality of energy pulses.

14. The apparatus of claim 8, wherein the energy pulse has an output power greater than 50 watts and the energy pulse has a duration less than 1 second.

15. The apparatus of claim 8, wherein the plurality of energy pulses are defined to cause necrosis of the portion of the lumen.

16. The apparatus of claim 8, wherein the expandable member is changed from the contracted configured to the expanded configuration when a fluid is injected into the expandable member.

17. The apparatus of claim 8, wherein each energy pulse from the plurality of energy pulses is an electromagnetic radiation pulse that has a spectral region corresponding to a microwave.

18. The apparatus of claim 8, wherein the portion of the lumen substantially surrounds the energy-emitting source.

19. A method, comprising:
compressing a portion of a prostatic urethra using an expandable member;
emitting an electromagnetic radiation pulse toward the compressed portion based on a pulse parameter value; and
modifying the pulse parameter value based on a signal from a sensor.

20. The method of claim 19, wherein the signal corresponds with a measurement of the compressed portion, the signal being defined during a rest period after the emitting.

21. The method of claim 19, wherein the electromagnetic radiation pulse is a first electromagnetic radiation pulse having a spectral region corresponding with a microwave,
the method further comprising:
emitting a second electromagnetic radiation pulse based on the pulse parameter value after the modifying.

22. The method of claim 19, wherein the indicator is associated with an impedance of the portion of the prostatic urethra.

23. The method of claim 19, wherein the emitting includes emitting from an electromagnetic radiation source, the signal is a first signal, at least a portion of the electromagnetic radiation source is configured to function as the sensor,
the method comprising:
receiving after the emitting a second signal at the portion of the electromagnetic radiation source, the first signal is defined based on the second signal.

24. The method of claim 19, wherein the indicator is associated with a temperature measurement from a rectal probe, the pulse parameter value is an output power value, the output power value is increased based on the indicator.
